(19) United States
(12) Patent Application Publication
(10) Pub. No.: US 2016/0340647 A1
(43) Pub. Date: Nov. 24, 2016

(54) COMPOSITIONS AND METHODS FOR INDUCING DIFFERENTIATION OF STEM CELLS

(71) Applicant: BIODEMAK LLC, Hoboken, NJ (US)

(72) Inventors: Miguel LOPEZ-TOLEDANO, Wellington, FL (US); Herbert WEISSBACH, Boynton Beach, FL (US); Shailaja KESARAJU, Boynton Beach, FL (US)

(21) Appl. No.: 15/111,954

(22) PCT Filed: Dec. 30, 2014
(86) PCT No.: PCT/US14/72651

§ 371 Date: Jul. 15, 2016

Related U.S. Application Data
(60) Provisional application No. 61/931,904, filed on Jan. 27, 2014.

(51) Int. Cl. C12N 5/0793; A61K 33/36; A61K 33/24; A61K 31/327; A61K 31/495; A61K 31/337; A61K 31/4745; A61K 31/7048; A61K 31/519; A61K 31/7068; A61K 31/513; C12N 5/09; A61K 31/664

(52) U.S. Cl. CPC C12N 5/0619; C12N 5/0693; A61K 33/36; A61K 33/24; A61K 31/327; A61K 31/495; A61K 31/664; A61K 31/4745; A61K 31/7048; A61K 31/519; A61K 31/7068; A61K 31/513; A61K 31/337; C12N 2506/08; C12N 2506/30; C12N 2501/999

(57) ABSTRACT

Compositions and methods for inducing differentiation of stem cells such as cancer stem cells (CSCs), and increasing sensitivity of CSCs to at least one oxidizing agent in a subject include sulindac and/or epimers thereof. These sulindac-based compositions and methods are particularly useful for treating cancers such as glioblastoma (GBM) and for differentiating stem cells in vitro that can be used for cell replacement therapy in a subject in need thereof.

### CROSS-REFERENCE TO RELATED APPLICATIONS

This application is a 371 National Stage Entry of International Application No. PCT/US14/72651 filed Dec. 30, 2014, which claims the benefit of Provisional Application Ser. No. 61/931,904, filed Jan. 27, 2014, both of which are hereby incorporated by reference their entireties, for all purposes, herein.

### FIELD OF THE INVENTION

The invention relates generally to the fields of chemistry, oncology, neurology and cellular therapy. More particularly, the invention relates to compositions and methods for inducing differentiation of stem cells such as neural stem cells (NSCs) and cancer stem cells (CSCs), and increasing sensitivity of CSCs to oxidative stress and oxidizing agents in a subject.

### BACKGROUND

Glioblastoma (GBM) is a very aggressive brain cancer representing 20% of all human intracranial tumors, with patients having a median survival time of less than 14 months. The treatments used have failed largely due to a high rate of tumor recurrence. GBM tumors are very heterogeneous, being comprised of several cell subtypes, including glioblastoma stem cells (GSCs). A subset of these cells retains the ability to repopulate the whole tumor when transplanted into mice (Hemmati H D, et al. (2003) Proc Natl Acad Sci USA 100(25):15178-15183). One of the most prevalent theories to explain the high tumor recurrence is the CSC theory, which proposes 1) tumors contain a number of cells that retain key stem cell properties and 2) tumorigenic cells arise from the transformation of tissue stem cells. According to this theory, GBMs arise from mutations that transform normal neural stem cells (NSCs) into GSCs, which are highly resistant to oxidative stress and anti-cancer therapies. There is a clear relationship between the appearance of GBM in NSC regions and its invasive and malignant features, supporting the theory that a specific transformation from a normal NSC to a GSC is involved in tumor initiation. GSCs are more resistant to traditional tumor treatments and they could be responsible for repopulating the heterogeneous population in the GBM, which would explain the high recurrence of tumors. Thus, GSCs appear to be an excellent target to prevent tumor reappearance. Drugs that can kill tumor cells without being toxic to normal cells and avoid cancer relapse by eliminating all possible remaining CSCs are needed.

### SUMMARY

Described herein are compositions and methods for inducing differentiation of stem cells (e.g., NSCs, CSCs), for increasing sensitivity of CSCs to oxidative stress and one or more oxidizing agents, and for increasing killing of CSCs caused by administration of an oxidizing agent. In one embodiment, the compositions and therapies are used for preventing GBM recurrence that include a) inhibiting the NSC to GSC transformation, b) eliminating the GSCs and c) making them more sensitive to anti-cancer treatment. Compositions for inducing differentiation of stem cells (e.g., NSCs, CSCs), for increasing sensitivity of CSCs to oxidative stress and one or more oxidizing agents, and for increasing killing of CSCs caused by administration of an oxidizing agent, include sulindac, epimers of sulindac, metabolites, derivatives, analogs, and variants thereof. Administration of such a composition results in one or more of: inhibition of tumor growth, reduction of tumor size, inhibition of metastasis, reduction in the number of tumor cells, etc. Sulindac is a non-steroidal anti-inflammatory drug (NSAID). Herein it is shown that sulindac can protect normal astrocytes against oxidative stress, while making GBM cells more sensitive to oxidative stress. Unexpectedly, it was observed that sulindac, primarily the S epimer, is able to induce neuronal differentiation in both NSCs and GSCs. The differentiated NSCs are also protected from oxidative stress damage, whereas the differentiation of GSCs to less undifferentiated cancer cells by sulindac increases the sensitivity of these cells to agents that cause oxidative stress. The elucidation of the mechanisms involved in sulindac-induced cell differentiation described herein provides for the development of specific drugs to prevent tumor recurrence. The cell differentiation and slow cell proliferation induced by sulindac on GSCs, and the altering of GSCs such that the GSCs demonstrate increased sensitivity to drugs, makes sulindac a desirable drug for slowing tumor growth. The sulindac S epimer shows the same properties as sulindac with respect to differentiation and enhanced killing, while the sulindac R epimer increases GSC sensitivity to oxidizing drugs.

Accordingly, described herein is a method of inducing differentiation of stem cells in vitro. The method includes culturing stem cells in the presence of sulindac or a sulindac epimer under conditions such that the stem cells differentiate into differentiated cells that can be transplanted into a subject (e.g., human) in need thereof. In one embodiment, the stem cells are cultured in the presence of a sulindac epimer and the sulindac epimer is an S epimer of sulindac. The stem cells can be NSCs and differentiate into neurons. In the method, the differentiated cells are suitable for use in cell replacement therapy in a subject (e.g., human) in need thereof. In one embodiment, the differentiated cells are neurons, and the subject suffers from a neurodegenerative disease.

Also described herein is a method of inducing differentiation of CSCs. The method includes delivering to a population of cells including CSCs a therapeutically effective amount of sulindac or a sulindac epimer for inducing differentiation of the CSCs into cancer cells that are sensitive to oxidative stress. The population of cells can further include NSCs, and in this embodiment, the NSCs are protected from oxidative stress. In one embodiment, the CSCs are GSCs. In other embodiments, the CSCs can be, for example, lung, skin, breast, liver, intestinal, colorectal, pancreatic or prostate CSCs.

Further described herein is a method of increasing sensitivity of CSCs to at least one oxidizing agent or agent that leads to the generation of reactive oxygen intermediates (ROS). The method includes delivering to a population of cells including CSCs a therapeutically effective amount of sulindac or a sulindac epimer for increasing sensitivity of the CSCs to the at least one oxidizing agent. In one embodiment of the method, a sulindac epimer is administered, and the sulindac epimer is an R epimer of sulindac. The at least one oxidizing agent or agent that leads to the generation of ROS can be one of, for example, As₂O₃, DOX, TBHP, DCA, temozolomide, cisplatin, cyclophosphamide, camptothecin, etoposide, vincristine, methotrexate, gemcitabine, 5-fluorouracil and paclitaxel. The population of cells can further include NSCs, and in this embodiment, the NSCs are protected from oxidative stress. In one embodiment, he CSCs are GSCs. In another embodiment, the CSCs are, for example, lung, skin, breast, liver, intestinal, colorectal, pancreatic or prostate CSCs.

Still further described herein is a method of increasing killing of CSCs caused by administration of an oxidizing agent or agent that leads to the generation of ROS. The method includes administering to a population of cells including CSCs a therapeutically effective amount of sulindac or a sulindac epimer for increasing sensitivity of the CSCs to the oxidizing agent or agent that leads to the generation of ROS prior to, concomitant with, or subsequent to administration of the oxidizing agent or agent that leads to the generation of ROS to the CSCs. The oxidizing agent or agent that leads to the generation of ROS can be one of, for example, As₂O₃, DOX, TBHP, DCA, temozolomide, cisplatin, cyclophosphamide, camptothecin, etoposide, vincristine, methotrexate, gemcitabine, 5-Fluorouracil and paclitaxel. The population of cells can further include NSCs, and in this embodiment, the NSCs are protected from oxidative stress. In one embodiment, the CSCs are GSCs. In another embodiment, the CSCs are lung, skin, breast, liver, intestinal, colorectal, pancreatic or prostate CSCs.

Unless otherwise defined, all technical terms used herein have the same meaning as commonly understood by one of ordinary skill in the art to which this invention belongs.

As used herein, “sulindac” refers to sulindac, both R and S epimers, sulindac derivatives, metabolites, analogues and variants thereof. Examples of sulindac metabolites include sulindac sulfide and sulindac sulfone.

As used herein, a “pharmaceutical salt” includes, but is not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

The compounds of the invention encompass various isomeric forms. Such isomers include, e.g., stereoisomers, e.g., chiral compounds, e.g., diastereomers and enantiomers.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not minor images of one another.

The term “enantiomers” refers to two stereoisomers of a compound which are non-superimposable mirror images of one another. An equimolar mixture of two enantiomers is called a “racemic mixture” or a “racemate.”

The terms “isomers” and “stereoisomers” refer to compounds which have identical chemical constitution, but differ with regard to the arrangement of the atoms or groups in space. Diastereoisomers that have the opposite configuration at only one of two or more tetrahedral stereogenic centers present in the respective molecular entities are known as “epimers.” Thus, when used herein “sulindac” or variants, derivatives or oxides thereof, includes epimeric and enantiomeric molecules.

With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

The terms “specific binding” and “specifically binds” refer to that binding which occurs between such paired species as enzyme/substrate, receptor/agonist, antibody/antigen, etc., and which may be mediated by covalent or non-covalent interactions or a combination of covalent and non-covalent interactions. In particular, the specific binding is characterized by the binding of one member of a pair to a particular species and to no other species within the family of compounds to which the corresponding member of the binding member belongs.

By the term “conjugated” is meant when one molecule or agent is physically or chemically coupled or adhered to another molecule or agent.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain (e.g., GBM), breast, pancreas, cervix, colon, head & neck, kidney, lung, non-small cell lung, melanoma, mesothelioma, ovary, sarcoma, stomach, uterus and Medulloblastoma. The term “cancer” includes any cancer arising from a variety of chemical, physical, and infectious organism cancer-causing agents.

The terms “therapeutic compound” and “active therapeutic agent” as used herein refer to a compound or agent useful in the prophylaxis or treatment of cancer.

The phrases “isolated,” “biologically pure,” and “chemically pure” refer to material which is substantially or essentially free from components which normally accompany it as found in its native state.

The expression “biologically compatible form suitable for administration in vivo” as used herein means a form of the substance to be administered in which any toxic effects are outweighed by the therapeutic effects. The substances may be administered to any subject, e.g., humans.

By “glioblastoma stem cells” and “GSCs” is typically meant cancer cells found within or obtained from glioblastoma tumors that possess characteristics associated with normal stem cells. GSCs are very resistant to oxidative damage and have the ability to self-renew and differentiate to any kind of more specialized non-stem cancer cell found in glioblastoma tumors. The differentiated non-stem cancer cells are more sensitive to oxidative stress damage. Additionally, these cells are characterized by being able to form neurosphere-like structures and the expression of certain cell surface markers, which include but are not limited to CD133 (prominin 1), SSEA-1 (stage-specific embryonic antigen-1), EGFR (epidermal growth factor receptor) and CD44 (homing cell adhesion molecule).

When referring to stem cells, in general, what is meant is undifferentiated biological cells that can differentiate into specialized cells and can divide (through mitosis) to produce more stem cells. When referring to “inducing differentiation of stem cells” and “such that the stem cells differentiate into differentiated cells” what is meant by “differentiated cells” are cells that possess a more distinct form and function than the stem cells from which they differentiated. For example, NSCs are undifferentiated, self-renewing, multipotent cells that can differentiate to the main cell phenotype of the central nervous system. By inducing their differentiation, NSCs become one of the more specialized cells, either astrocytes, oligodendrocytes or neurons. In some cases, specific treatments can drive NSC differentiation toward a specific phenotype, e.g., neurons. Specific markers can then be used to recognize NSC (e.g., nestin, mushashi or EGFR), neurons (e.g., beta-tubulin III, MAP-2, NeuN, Neurofilament), astrocytes (e.g., GFAP, S100) and oligodendrocytes (e.g., GAL-C, NG2, 01). In addition to the normal differentiation of NSC (and stem cells in general), the Cancer Stem Cell Theory proposes that cancer stem cells arise from normal stem cells. Any possible treatment that could both prevent this conversion or make the newly formed cancer stem cell more susceptible to death would prevent tumor initiation and/or growth and is encompassed by the present invention.

By the phrase “differentiated cells are suitable for use in cell replacement therapy” is meant differentiated cells that have the ability to be grafted in the NSC, migrate within the brain, incorporate into the existing circuitry and regain the function that has been lost due to the previous neuronal degeneration. Cell replacement therapy with stem cells has been used in different studies, focusing on specific neurodegenerative diseases that result from neuronal loss, including Parkinson's disease, Huntington's disease, Alzheimer's disease, amyothrophic lateral sclerosis and spinal muscular atrophy.

As used herein, the terms “diagnostic,” “diagnose” and “diagnosed” mean identifying the presence or nature of a pathologic condition (e.g., GBM).

By the phrase “immune response” is meant induction of antibody and/or immune cell-mediated responses specific against an antigen, antigens, cancer cell, pathogen, pathogenic agent, etc.

By the phrases “therapeutically effective amount” and “effective dosage” is meant an amount sufficient to produce a therapeutically (e.g., clinically) desirable result; the exact nature of the result will vary depending on the nature of the disorder being treated. The compositions described herein can be administered from one or more times per day to one or more times per week. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient” “subject” and “individual” are used interchangeably herein, and mean an animal to be treated, including vertebrates and invertebrates. Typically, a subject is a human. In some cases, the methods of the invention find use in experimental animals, in veterinary applications (e.g., equine, bovine, ovine, canine, feline, avian, etc.), and in the development of animal models for disease, including, but not limited to, rodents including mice, rats, and hamsters, as well as non-human primates.

The term “sample” is used herein in its broadest sense. A sample may include a bodily fluid, a soluble fraction of a cell preparation or media in which cells were grown, genomic DNA, RNA or cDNA, a cell, a tissue, skin, hair and the like. Examples of samples include saliva, serum, blood, urine and plasma.

Although compositions, kits and methods similar or equivalent to those described herein can be used in the practice or testing of the present invention, suitable compositions, kits, and methods are described below. All publications, patent applications, and patents mentioned herein are incorporated by reference in their entirety. For example, U.S. Pat. Nos. 8,765,808, 8,603,985, and 8,357,720 are incorporated by reference herein in their entirety. In the case of conflict, the present specification, including definitions, will control. The particular embodiments discussed below are illustrative only and not intended to be limiting.

### BRIEF DESCRIPTION OF THE DRAWINGS

FIG. 1 shows micrographs of cells in culture. A) NSCs grown in culture in the presence of EGF+bFGF as undifferentiated floating clusters of cells called neurospheres. B) U87 glioblastoma cell line. C) Floating GBM stem cell neurospheres (GSC) derived from U87 cells grown in the presence of EGF+bFGF

FIG. 2 shows micrographs of cells in culture and percentage of cell types at different days post plating (dpp). A) NSCs grown in culture in the presence of EGF+bFGF as undifferentiated floating clusters of cells called neurospheres. B) When plated on poly-L-lysine (PLL), NSCs spontaneously differentiate into neurons β-tubulin III+), astrocytes (GFAP+) and oligodendrocytes (01+). C) Percentage of proliferating cells (Ki67+) vs total number of cells at dpp. D) Percentage of NSCs (Nestin+) vs total number of cells at different dpp. E) Percentage of neurons βTubulin III+) vs total number of cells at different dpp. F) Percentage of astrocytes (GFAP+) vs total number of cells at different dpp.

FIG. 3 shows that sulindac induces cell differentiation in NSCs. Immunocytochemistry with the neuronal-specific antibody beta-tubulin III and NSC-specific antibody nestin was performed at different time points, counterstained with the nuclear stain hoechst. Floating NSCs were treated for 24 hours with vehicle (A) or 500 microMolar sulindac and plated on PLL for another 24 hours (B) or PLL-plated NSC and treated for 48 hours with vehicle (C) or 500 microMolar sulindac at the 7th day post-plating (D). The total number of neurons was quantified and normalized vs. non-treated cultures for floating cells (E) and plated cells (F). Under both conditions, NSC progeny showed higher neuronal differentiation with the sulindac treatment. The graphs represent the percentage of neurons vs. the control. **p<0.01; ***p<0.001.

FIG. 4 presents evidence that sulindac induces cell differentiation of GSCs. Floating GSCs were treated for 24 hours with vehicle or 500 uM sulindac and plated on PLL for another 24 hours, for two days with 500 μM sulindac after one dpp. (B) or for five days with 500 μM sulindac after three dpp (D). In all cases, sulindac induced a clear morphological differentiation vs. their controls (A and C). The western blot (E) and densitometry against nestin (F), (G), DCX (H) and actin support the data showing a higher neuronal differentiation of GSC after sulindac treatment

FIG. 5 shows that sulindac enhances cancer cell killing while protecting non-tumoral cells from oxidative stress by TBHP. A) NSCs were treated as floating neurospheres for 24 hours with vehicle or sulindac and plated on PLL for two hours before assaying for cell viability. B) NSCs were plated on PLL, differentiated for five days, followed by 48 hours treatment with vehicle or sulindac. C) Astrocytes were plated on PLL for seven days, followed by 48 hours treatment with vehicle or sulindac. D) GSC were treated as floating neurospheres for 24 hours with vehicle or sulindac and plated on PLL for two hours before assaying for cell viability. E) GSC were plated on PLL and differentiated for five days, followed by 48 hours treatment with vehicle or sulindac. F) U87 glioblastoma cells were plated for seven days, followed by 48 hours treatment with vehicle or sulindac. NSCs (A and B) were obtained from mice hippocampi and grown in defined medium for neurospheres. Astrocytes (C) were purified by differentiating NSCs (95-100% GFAP+ cells) followed by maintenance in complete medium. GSC (D and E) were purified from the U87 GBM cell line (F). All cell cultures were plated on PLL-coated 96-well plates. All treatments were either 500 μM sulindac (triangles) or vehicle (squares). Cell viability was determined with the CellTiter 96 Aqueous One Cell Proliferation Assay (Promega) after the addition of different concentrations of TBHP, performed in quadruplicate, for two hours (Error bar: SEM). The graphs represent the percentage of cell survival vs. the control without TBHP treatment.

FIG. 6 shows that the S epimer of sulindac is responsible for inducing cell differentiation of GSCs. Following treatment 2, GSCs were treated for two days with A) vehicle, B) 500 uM sulindac, C) 25 uM of sulindac suphone, D) 400 uM of ibuprofen, E) 250 uM of the R epimer of sulindac or F) 250 uM of the S epimer of sulindac after one dpp.

FIG. 7 shows that sulindac epimers enhance cancer killing in combination with different oxidizing agents. Following treatment 2, GSC were treated for 48 hours treatment with vehicle (control), 500 uM sulindac (Sul), 250 uM of the S epimer of sulindac (SulS) or 250 uM of the R epimer of sulindac (SulR). The cells were also treated with A) 200 mM TBHP B), 3 mM As₂O₃, C) 30 mM DCA and D) 400 nM Doxorubicin (DOX).

FIG. 8 shows the effect of sulindac on RTP801 levels in both U87 cells and GSCs. However, sulindac produces an increase in GSC at 0+72 (both floating and plated), 0+3 and 0+6. Sulindac when added at the 3^{rd} dpp, induced a decrease in RTP801. This results are consistent with the pattern of differentiation shown in NSC and in vivo, as reported in the paper Malagelada et al, 2011 (3186 •The Journal of Neuroscience, Mar. 2, 2011 •31(9):3186-3196).

### DETAILED DESCRIPTION

Described herein are compositions including sulindac and/or epimers thereof, and methods involving use of sulindac and/or epimers thereof, for inducing differentiation of stem cells such as NSCs and CSCs, and increasing sensitivity of CSCs to oxidative stress and one or more (e.g., at least one) oxidizing agents in a subject. These sulindac-based compositions and methods are particularly useful for treating cancers such as GBM and for differentiating stem cells in vitro that can be used for cell replacement therapy in a subject in need thereof. Sulindac has a chiral sulfur center so it contains an equal mixture of the S and R epimers. The use of sulindac to complement chemotherapy because of its ability to differentiate GSC has advantages compared with other known compounds. Sulindac is inexpensive, it has been used in the clinic for years with low toxicity, and it appears to be able to penetrate the blood brain barrier. In addition, sulindac enhances cancer cell's killing due to increased oxidative stress (Ayyanathan et al., (2012) PLoS One 7(7):e39949; Marchetti M, et al. (2009) PLoS One 4(6):e5804) and exerts a protective effect on normal cells (Moench et al. (2009) Proc Natl Acad Sci USA 106(46):19611-19616). As noted above, CSCs are more resistant to chemotherapy than the cancer cells that are derived after differentiation. In the experiments described herein, what effect sulindac might have when normal and cancer stem cells are exposed to oxidative stress was examined. For these studies, the effect of sulindac on normal astrocytes, NSC, a GBM cell line (U87), and GSC, after exposure to oxidizing agents or anticancer drugs that affect mitochondrial respiration, was examined. These studies support the protective role of sulindac on normal cells (both mature astrocytes and NSC) and its ability to enhance the sensitivity of GBM cells to oxidative stress. An important new finding is that sulindac induces differentiation of both NSCs and GSCs, and that the GSC-derived cancer cells show enhanced sensitivity to oxidative stress. The compositions described herein provide for administration of a lower dose of an anti-cancer therapeutic agent (e.g., chemotherapy). A benefit of lowering the dose of the anti-cancer agent administered to a subject includes a decrease in the incidence of adverse effects associated with higher dosages, a reduction in the administration of analgesic agents needed to treat pain associated with the adverse effects, an improvement in patient compliance, etc.

The below described preferred embodiments illustrate adaptations of these compositions and methods. Nonetheless, from the description of these embodiments, other aspects of the invention can be made and/or practiced based on the description provided below.

### Compositions for Inducing Differentiation of Stem Cells and Increasing Sensitivity of CSCs to Oxidative Stress and Oxidizing Agents

A typical composition for inducing differentiation of stem cells in vitro, for inducing differentiation of NSCs or CSCs, for increasing sensitivity of CSCs to oxidative stress and one or more oxidizing agents and for increasing killing of CSCs caused by administration of an oxidizing agent includes sulindac or a sulindac epimer (also referred to herein as “an epimer thereof”), or a metabolite, derivative, analog, or variant thereof. The composition can consist solely of a therapeutically effective dose of sulindac or an epimer thereof, or can include a therapeutically effective dose of sulindac or an epimer thereof as well as a pharmaceutically acceptable carrier. Such a composition can further include at least one oxidizing agent as described below.

A typical composition for inducing differentiation of stem cells in vitro includes a therapeutically effective amount of sulindac or an epimer thereof for inducing differentiation of the stem cells. In a particular embodiment, the stem cells are NSCs that are differentiated into neurons. The resultant differentiated cells are suitable for transplantation into a subject in need thereof. In general, the concentration of sulindac is in the range of approximately 100-800 μM, and if the composition consists of the sulindac S epimer, the concentration of the S epimer is in the range of approximately 50-500 μM, and if the composition consists of the sulindac R epimer, the concentration of the R epimer is in the range of approximately 50-500 μM. In one embodiment, this composition is added to culture medium containing cells.

Similarly, a typical composition for inducing differentiation of CSCs in a human subject suffering from cancer, for example, includes a therapeutically effective amount (dose) of sulindac or a sulindac epimer for inducing differentiation of the CSCs into cancer cells that are sensitive to oxidative stress. The composition can consist solely of a therapeutically effective dose of sulindac or an epimer thereof, or can include a therapeutically effective dose of sulindac or an epimer thereof as well as a pharmaceutically acceptable carrier. In one embodiment, the composition includes the sulindac S epimer, and in another embodiment, the composition includes the sulindac R epimer.

A composition for increasing sensitivity of CSCs to at least one oxidizing agent (e.g., in a human subject suffering from GBM) includes a therapeutically effective amount of sulindac or a sulindac epimer for increasing sensitivity of the CSCs to at least one oxidizing agent. In one embodiment, the composition includes the sulindac S epimer, and in another embodiment, the composition includes the sulindac R epimer. In an embodiment in which the composition is administered for increasing sensitivity of CSCs to at least one oxidizing agent and/or increasing killing of CSCs caused by administration of an oxidizing agent, the composition typically includes a therapeutically effective dose of sulindac or epimer thereof.

In a typical embodiment, the therapeutically effective amount of sulindac or a sulindac epimer described herein generally ranges from about 20 mg to about 400 mg for an approximately 70 kg patient.

In the compositions described herein, sulindac can be obtained commercially or synthesized. Methods for producing sulindac, its epimers and derivatives thereof, as well as therapeutic formulations thereof, are described in U.S. Pat. Nos. 8,765,808, 8,603,985, and 8,357,720, all incorporated herein by reference. A commercial source for purchasing sulindac is Sigma (Sigma-Aldrich™ St. Louis, Mo.). For the sulindac epimers, including bulk amounts, the Regis Technologies company (Regis Technologies, Inc. Morton Grove, Ill.) is a suitable commercial source.

In one embodiment, a composition including sulindac or an epimer thereof can be a combination of two or more agents, i.e., a first agent that is sulindac, the R epimer of sulindac (R sulindac) or the S epimer of sulindac (S sulindac) or their metabolites or derivatives, and a second agent that is an oxidizing agent or agent that leads to the generation of (e.g., produces) reactive oxygen intermediates or ROS. Agents that produce ROS in cells can be used for the treatment of internal cancers, for example, while oxidizing agents may be used topically, for example. The oxidizing agent or agent that leads to the generation of reactive oxygen intermediates or ROS can be in any amount which is sufficient to induce reactive oxygen species intracellularly or extracellularly, in vitro or in vivo. Such an agent is typically present at a concentration that varies depending on the drugs. In a typical embodiment, 0.1-1 times the normal therapeutic dose is used. In an embodiment in which a composition includes sulindac, the sulindac R epimer or the sulindac S epimer (or their metabolites or derivatives) and an oxidizing agent or agent which induces ROS, the agent can be any suitable such agent, for example, known anti-cancer drugs such as As₂O₃, dichloroacetic acid (DCA), cisplatin, vincristine, doxorubicin, etc., that have been shown to affect mitochondrial function and produce ROS. The combination of sulindac, or the sulindac epimers, with these agents can be used for the treatment of internal cancers since these drugs can be administered orally or by injection or infusion. Oxidizing agents, such as peroxides, nitrates, hypochlorites, etc., including hydrogen peroxide, superoxide, tert-butylhydroperoxide, peroxynitrites, hypochlorous acid, etc., when used in combination with sulindac or its epimers can be administered using topical formulations for treatment of skin lesions, both precancerous and cancerous since these oxidizing agents cannot be administered internally.

As used herein, the terms peroxide and peroxide compound are meant to include hydrogen peroxide, inorganic peroxides, organic peroxides, peroxide complexes, other compounds containing the peroxy (peroxy) —O—O— moiety, superoxides, and peroxide precursor compounds which generate peroxide species in situ. Examples of organic peroxides include hydroperoxides, internal peroxides, endoperoxides, diacyl peroxides, ketone peroxides, peroxydicarbonates, peroxyesters, dialkyl peroxides, peroxyketals, and peroxyacids. Methods for the synthesis of organic peroxides are well known to those of skill in the art and can be used in accordance with the present invention. Commercially available peroxides and peroxide compounds, as well as methods of synthesis of peroxides and peroxide compounds, are disclosed in U.S. Pat. Nos. 8,765,808, 8,603,985, and 8,357,720.

When administered as a combination, the therapeutic agents can be formulated as separate compositions which are given at the same time or different times, or the therapeutic agents can be given as a single composition.

The compositions, methods, and kits described herein have both prophylactic and treatment applications, i.e., can be used as a prophylactic to prevent onset of a disease or condition in a subject, as well as to treat a subject having a disease or condition.

### Methods of Inducing Differentiation of Stem Cells, Increasing Sensitivity of CSCs to Oxidative Stress and an Oxidizing Agent and Increasing Killing of CSCs Caused by Administration of an Oxidizing Agent

Methods of inducing differentiation of stem cells (e.g., CSCs, NSCs, etc.), increasing sensitivity of CSCs to oxidative stress and at least one oxidizing agent, and increasing killing of CSCs caused by administration of an oxidizing agent all include use of sulindac or a metabolite, derivative or analogue thereof, or a sulindac epimer or a metabolite, derivative or analogue thereof. The methods can be used for culturing differentiated cells that can be used in cell replacement therapy in a subject in need thereof (i.e., transplanted into the subject to treat a disease or disorder). A method of inducing differentiation of stem cells in vitro includes culturing stem cells in the presence of sulindac or a sulindac epimer under conditions such that the stem cells differentiate into differentiated cells that can be transplanted into a subject in need thereof. In one example of this method, the S epimer of sulindac is used. In the experiments described below, the S epimer induced neuronal differentiation in both NSCs and GSCs. In a particular embodiment, the stem cells are NSCs and are differentiated into neurons. Such neurons can be transplanted into a subject (e.g., a human subject) suffering from a neurodegenerative disease that involves neuronal loss such as Parkinson's disease, Huntington's disease, Alzheimer's disease, amyothrophic lateral sclerosis, Picks disease and spinal muscular atrophy.

Any suitable conditions for differentiating stem cells into differentiated cells (e.g., neurons) that can be transplanted into a subject can be used. Several different kinds of stem cells can be used for specific neuronal differentiation. These include embryonic stem (ES) cells, tissue specific stem cells, progenitor cells, mesenchymal stem cells (MSCs), and induced pluripotent stem (iPS) cells. Such conditions are known in the art, and are described in J. Simon Lunn et al., Ann Neurol. September 2011; 70(3): 353-361; Hynek Wichterle et al., Cell, Volume 110, Issue 3, Aug. 9, 2002; U.S. Pat. No. 7,390,659; and U.S. Pat. No. 8,426,200. Existing commercial culture conditions for neuronal differentiation includes NeuroCult™ NS-A Differentiation Kit (Human), from Stem Cells Technologies. To identify neuronal differentiation, specific cell markers displayed by neurons can be used. Those include beta-tubulin III, Microtuble-associated protein 2 (MAP-2), Neuronal nuclei antigen (NeuN), Neuron Specific Enolase (NSE) and Neurofilament. For specific neuronal phenotypes, markers like Tyrosine hydroxylase (TH) for dopaminergic neurons, Choline Acetyltransferase (ChAT) expressed in cholinergic neurons, Calbindin in cereberal Purkinje cells and granule cells of hippocampus or Gamma-Aminobutyric Acid (GABA) in inhibitory GABAergic neurons can be used.

In other embodiments, the methods can be used for differentiating CSCs into cancer cells that are sensitive to oxidative stress, for example, cancer cells that are sensitive to at least one oxidizing agent. In such methods, a therapeutically effective amount of sulindac or a sulindac epimer for inducing differentiation of CSCs into cancer cells that are sensitive to oxidative stress is delivered to CSCs, for example, a population of cells that includes CSCs and cells that are not CSCs. In one embodiment, the sulindac or sulindac epimer is delivered to a population of cells that includes CSCs and NSCs such that the CSCs are differentiated into cancer cells sensitive to oxidative stress while the NSCs are protected from oxidative stress. The CSCs can be any cancer stem cell, including one or more of: GBM, lung, skin, breast, liver, colorectal, pancreatic and prostate CSCs. In an embodiment of the method for increasing sensitivity of CSCs to at least one oxidizing agent, a therapeutically effective amount of sulindac or a sulindac epimer for increasing sensitivity of the CSCs to the at least one oxidizing agent is delivered to CSCs, or a population of cells that includes CSCs and cells that are not CSCs. As with the method described above, the sulindac or sulindac epimer is delivered to a population of cells that includes CSCs and cells that are not CSCs (e.g., NSCs, noncancerous cells, normal cells) such that the CSCs are differentiated into cancer cells sensitive to oxidative stress while the cells that are not CSCs are protected from oxidative stress. In a particular embodiment, the R epimer of sulindac is used, as in the experiments described below, the R epimer was shown to alter GSCs such that the GSCs demonstrated increased sensitivity to oxidizing agents. In a similar embodiment, sulindac or an epimer thereof is used in a method of increasing killing of CSCs caused by administration of an oxidizing agent. This method includes administering to CSCs or a population of cells that includes CSCs and cells that are not CSCs a therapeutically effective amount of sulindac or a sulindac epimer for increasing sensitivity of the CSCs to the oxidizing agent. The sulindac or sulindac epimer can be administered prior to, concomitant with, or subsequent to administration of the oxidizing agent. As with the embodiments described above, cells that are not CSCs (e.g., NSCs, noncancerous cells, normal cells) are protected from oxidative stress. Suitable oxidizing agents for use in such methods are described above. A nonlimiting list of such agents includes: As₂O₃, DOX, TBHP, DCA, temozolomide, cisplatin, cyclophosphamide, camptothecin, etoposide, vincristine, methotrexate, gemcitabine, 5-fluorouracil and paclitaxel.

The therapeutic methods of the invention (which include prophylactic treatment) in general include administration of a therapeutically effective amount of the compositions described herein to a subject in need thereof, including a mammal, particularly a human Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., histological assay, genetic test, enzyme or protein marker, family history, and the like).

Additional cancers which can be treated by the disclosed compositions include but are not limited to, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

Inhibition of tumor cell growth refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

Administration of a composition as described herein generally results in no local adverse reactions in the subject. The compositions and methods described herein can be utilized with any suitable subject, including invertebrate and vertebrate subjects. In a typical embodiment, a subject to be treated is an animal such as a mammal (e.g., human beings, rodents, dogs, cats, goats, sheep, cows, horses, etc.). A human patient suffering from or at risk of developing cancer is a typical subject. For example, the subject is a human, and the cancer is one of: brain (e.g., GBM), lung, skin, breast, liver, colorectal, pancreatic and prostate cancer.

In one embodiment, the invention provides a method of monitoring treatment progress. The method includes the step of determining a level of changes in the tumor load by patient screening using physical exams, laboratory clinical tests, pathology reports and imaging technologies such as CT scan, MRI, ultrasound, etc. (e.g., standard assays such as for example, imaging, mechanical measurements, in vitro assays, etc.) in a subject suffering from or susceptible to a disorder or symptoms thereof associated with cancer (e.g., GBM) in which the subject has been administered a therapeutic amount of a composition as described herein. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

### Administration of Compositions

The compositions described herein may be administered to invertebrates, animals, and mammals (e.g., dog, cat, pig, horse, rodent, non-human primate, human) in any suitable formulation. For example, a composition including a therapeutically effective amount of sulindac or a sulindac epimer may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences (Mack Pub. Co., Easton, Pa., 17^{th} Edition, 1985), Remington: The Science and Practice of Pharmacy (by Loyd V. Jr et al., Pharmaceutical Press; 22nd Edition, 2012), standard texts in this field, and in USP/NF. Other substances may be added to the compositions to stabilize and/or preserve the compositions.

The compositions described herein may be administered to a subject (e.g., mammals) by any conventional technique. Typically, such administration will be parenteral (e.g., intravenous, subcutaneous, intramuscular, intraperitoneal, oral, nasal, or intrathecal introduction). The compositions may also be administered directly to a target site. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form. In therapeutic applications, the compositions described herein are administered to an individual suffering from cancer (e.g., GBM). In prophylactic applications, the compositions described herein are administered to an individual at risk of developing (e.g., genetically predisposed to) cancer.

The compositions described herein may be formulated for any suitable route of administration. The formulation and preparation of such compositions are well known to those skilled in the art of pharmaceutical formulation and may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the sulindac or sulindac epimer (active therapeutic agent), the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, compositions (e.g., pharmaceutical compositions) described herein may be in a form suitable for sterile injection. To prepare such a composition, the suitable active therapeutic agent(s) can be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., sulindac, the S epimer of sulindac, the R epimer of sulindac, a derivative of sulindac or a derivative of a sulindac epimer) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches such as potato starch, calcium carbonate, sodium chloride, lactose, calcium phosphate, calcium sulfate, or sodium phosphate); granulating and disintegrating agents (cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

In one embodiment, sulindac or a sulindac epimer is included in an eye drop formulation for treatment of an ophthalmic disorder, for example, retinoblastoma. Thus, the compositions described herein can be administered by intravitreal or other ophthalmic modes of administration.

Optionally, a composition as described herein may be administered in combination with any other appropriate therapy; such methods are known to the skilled artisan and described in Remington: The Science and Practice of Pharmacy, supra. For example, a composition as described herein can be administered in conjunction with one or more of surgery (any appropriate surgical intervention), radiotherapy (e.g., any mechanism for inducing DNA damage locally within tumor cells such as gamma-irradiation, X-rays, UV-irradiation, microwaves, electronic emissions, directed delivery of radioisotopes to tumor cells, etc.), cytokine therapy, and chemotherapy. A composition as described herein may be administered simultaneously with, before, or after surgery, radiation or chemotherapy treatment. Chemotherapeutic agents can be co-administered, precede, or administered after a composition as described herein. Non-limiting examples of chemotherapeutic agents include As₂O₃, dichloroacetic acid (DCA), TBHP, temozolomide, cisplatin, cyclophosphamide, camptothecin, etoposide, vincristine, methotrexate, gemcitabine, 5-fluorouracil, paclitaxel, bisphenol A (BPA), tetramethylrhodamine derivatives, N-(4-hydroxyphenyl)retinamide (HPR), dithiophene, menadione (vitamin K3) X radiation, or phytol (3,7,11,15-tetramethyl-2-hexadecene-1-ol). As will be understood by those of ordinary skill in the art, the appropriate doses of chemotherapeutic agents will be generally around those already employed in clinical therapies wherein the chemotherapeutics are administered alone or in combination with other chemotherapeutics. Some variation in dosage will necessarily occur depending on the condition of the subject being treated. The physician responsible for administration will be able to determine the appropriate dose for the individual subject. Combinations are expected to be advantageously synergistic. The general use of combinations of substances in cancer treatment is well known. In a method of treating cancer (e.g., inducing differentiation of CSCs, increasing sensitivity of CSCs to oxidizing agents or other anti-cancer agents, killing CSCs, reducing cancer tumor size, etc.) by employing a combined anti-tumor therapy, a composition as described herein is administered to a subject (e.g., a mammal having a GBM) in combination with another anti-cancer agent in a manner effective to result in their combined anti-tumor actions within the subject. The agents would therefore be provided in amounts effective and for periods of time effective to result in their combined presence in proximity to CSCs, within a tumor and/or tumor vasculature, etc., and their combined actions in proximity to CSCs, within a tumor and/or tumor vasculature, etc. In such an embodiment, the composition and other anti-cancer agent(s) may be administered to the subject simultaneously, either in a single composition, or as two distinct compositions using different administration routes. Alternatively, administration of a composition as described herein may precede, or follow, administration of the second anti-cancer agent treatment by, e.g., intervals ranging from minutes to weeks. In some embodiments, it may be desirable to extend the time period for treatment significantly, where several days (2, 3, 4, 5, 6 or 7), several weeks (1, 2, 3, 4, 5, 6, 7 or 8) or even several months (1, 2, 3, 4, 5, 6, 7 or 8) lapse between the respective administrations.

Kits for inducing differentiation of stem cells (e.g., CSCs) in a subject are also described herein. In a typical embodiment, a kit includes a therapeutically effective amount of sulindac or a sulindac epimer for inducing differentiation of stem cells such as, for example, CSCs. In another embodiment, a kit includes at least one of: sulindac, a variant or derivative thereof, the S epimer of sulindac, a variant or derivative thereof, an oxidizing agent (e.g., As2O3, DOX, TBHP, DCA, temozolomide, cisplatin, cyclophosphamide, camptothecin, etoposide, vincristine, methotrexate, gemcitabine, 5-fluorouracil, paclitaxel etc.), as well as printed instructions for using the composition to reduce the rate of tumor growth in a subject. In a kit, the composition may further include a pharmaceutically acceptable carrier in unit dosage form.

### Effective Doses

The compositions described herein are preferably administered to a subject (e.g., invertebrates, animals, mammals (e.g., dog, cat, pig, horse, rodent, non-human primate, human)) in an effective amount, that is, an amount capable of producing a desirable result in a treated subject (e.g., differentiation of CSCs, increased sensitivity of CSCs to an oxidizing agent or other anti-cancer agent, killing of CSCs, reduction of tumor growth, etc.). Such a therapeutically effective amount can be determined as described below.

Toxicity and therapeutic efficacy of the compositions described herein can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one subject depends on many factors, including the subject's size, body surface area, age, the particular composition to be administered, time and route of administration, general health, and other drugs being administered concurrently.

### EXAMPLES

The present invention is further illustrated by the following specific examples. The examples are provided for illustration only and should not be construed as limiting the scope of the invention in any way.

### Example 1

### Sulindac Induces Differentiation in NSCs and GSCs: Therapeutic Applications

### Results

Preparation of Various Cell Lines:

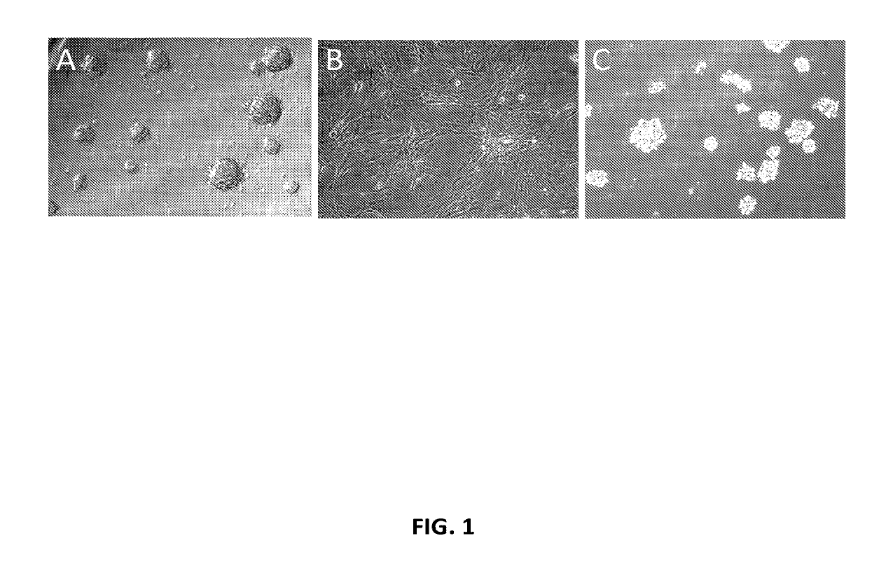

As previously reported, NSCs can grow almost indefinitely in culture in floating clusters of dividing cells called neurospheres (FIG. 1A). NSC stop proliferating and start differentiating after attaching to an adherent substrate (poly-L-lysine, PLL) and removal of the growth factors (EGF and bFGF). Under these circumstances, NSCs differentiate into neurons, astrocytes and oligodendrocytes. Pure astrocytes were isolated from NSCs, as described in methods. To isolate GSC, the U87 glioblastoma cancer cell line was used according to a previously published protocol (Yu S C, et al. (2008) Cancer letters 265(1):124-134). By growing U87 cells (FIG. 1B) in the presence of EGF and bFGF, GSCs grow forming floating clusters of cells, similarly to NSC neurospheres (FIG. 1C). Thus, under the conditions described, it is possible to compare the effect of sulindac on four cell lines: NSC, astrocytes, U87 and GSC under various conditions.

Effect of Sulindac on NSC and GSC Differentiation:

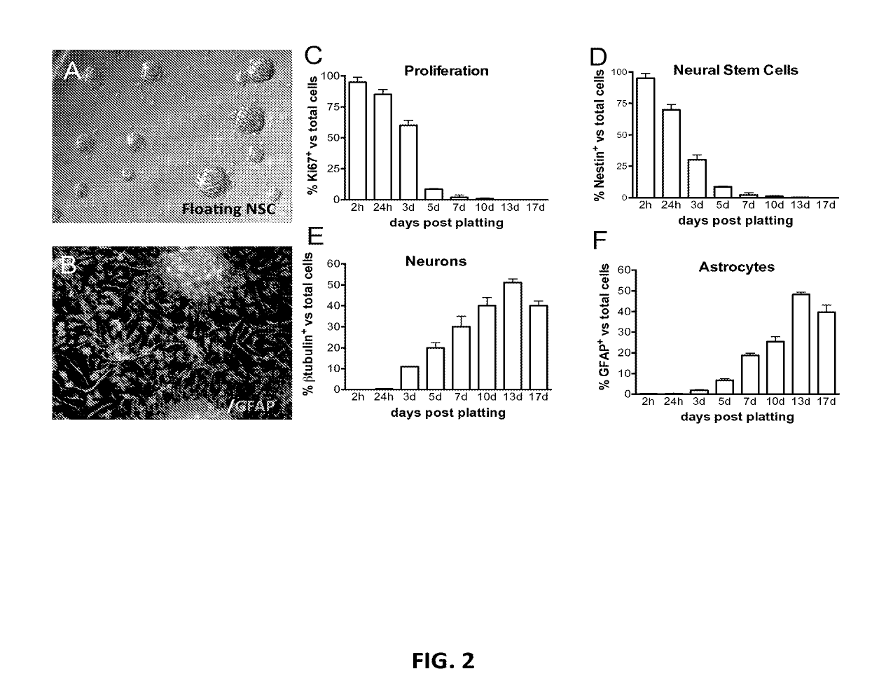

NSCs grow floating as neurospheres (FIG. 1A) and spontaneously differentiate after plating on PLL (FIG. 2B). The percentage of the different cell types in culture varies at different days post plating. Floating neurospheres at day zero are almost pure nestin⁺ NSC, whereas after the 7^{th} day post plating (dpp) the culture is a mix of mature neurons and astrocytes, with hardly detectable levels of oligodendrocytes and a mix of glial and neuronal progenitor cells (FIG. 2C, D, E, F). Because of this, two different treatments were selected (referred to as treatment 1 and treatment 2, see methods) using sulindac and NSC, based on the time post plating (either 1 or 7 days), which reflect the cell type present in the culture and the time of treatment with sulindac (1 or 2 days). To assure a fair comparison with GSC, a similar protocol for GSC was used, as described in methods.

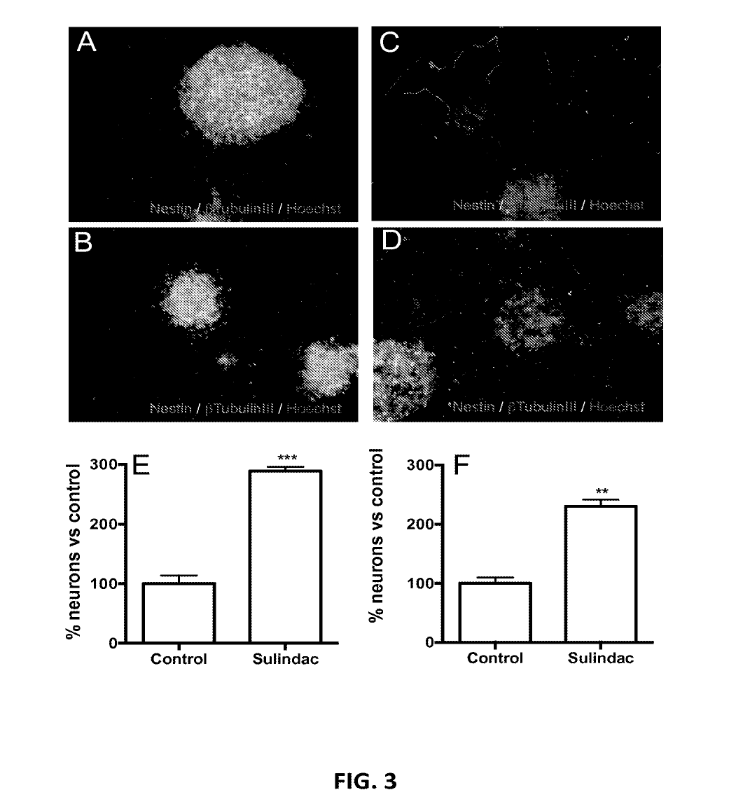

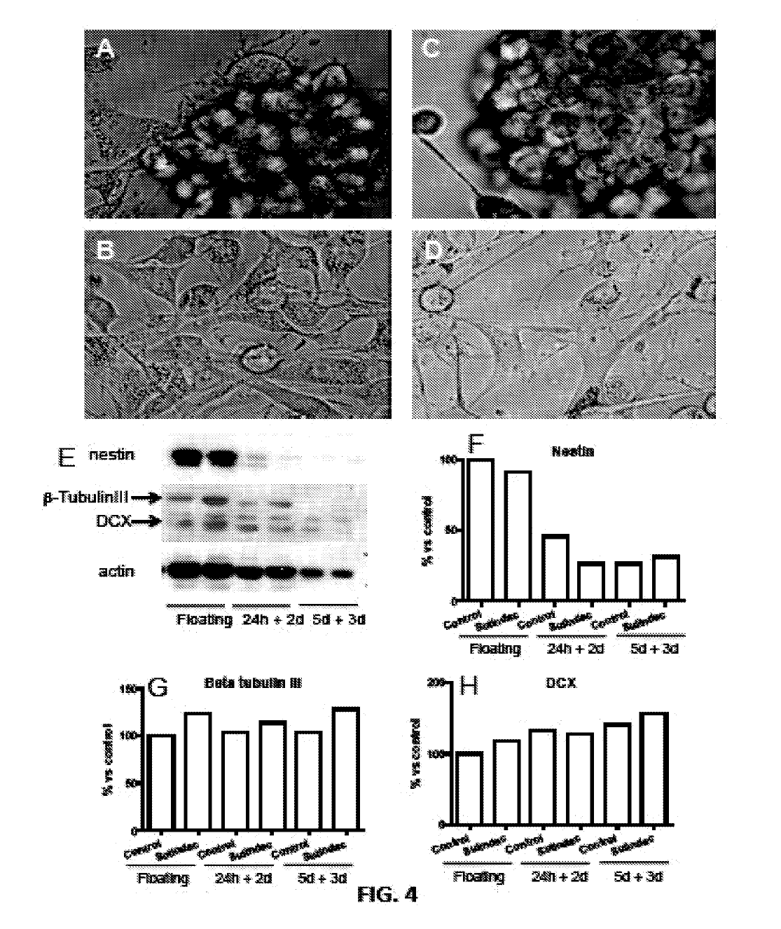

FIG. 3 shows the formation of neurons obtained after sulindac treatment of NSCs. FIG. 3E shows that sulindac treatment 1 of NSC induced a significant increase in the number of neurons (FIG. 3C) as compared with vehicle (FIG. 3A). A similar result was obtained with treatment 2 (FIG. 3F) between control (FIG. 3B) and cells treated with sulindac (FIG. 3D). The bar graphs show the percentage of neurons vs. control measured by immunocytochemistry against the neuronal-specific protein beta-III-tubulin, as explained in methods. Similarly to NSC, floating GSCs could also be disaggregated and forced to attach to PLL. After plating, some of the GSCs showed more differentiated features, however, a high percentage of GSCs remained growing as attached clusters and eventually detached and grew floating in the culture plate. This suggests that GSCs were lacking some cellular signals for differentiation that were present in NSCs. This is shown in FIG. 4A, in which GSCs were treated with vehicle (treatment 1). However, when GSC cells were exposed to sulindac (treatment 1), they showed a clear differentiation effect (FIG. 4B). Treatment 2 showed similar results (FIGS. 4C and 4D). Like NSC, the treatment with sulindac seems to particularly induce a neuronal phenotype on GSC (FIG. 4E). In summary, these results show that sulindac induces cell differentiation in both NSCs and GSCs at the times studied.

Effect of Sulindac on NSC, GSC, Astrocytes and Glioblastoma Cells Exposed to Oxidative Stress:

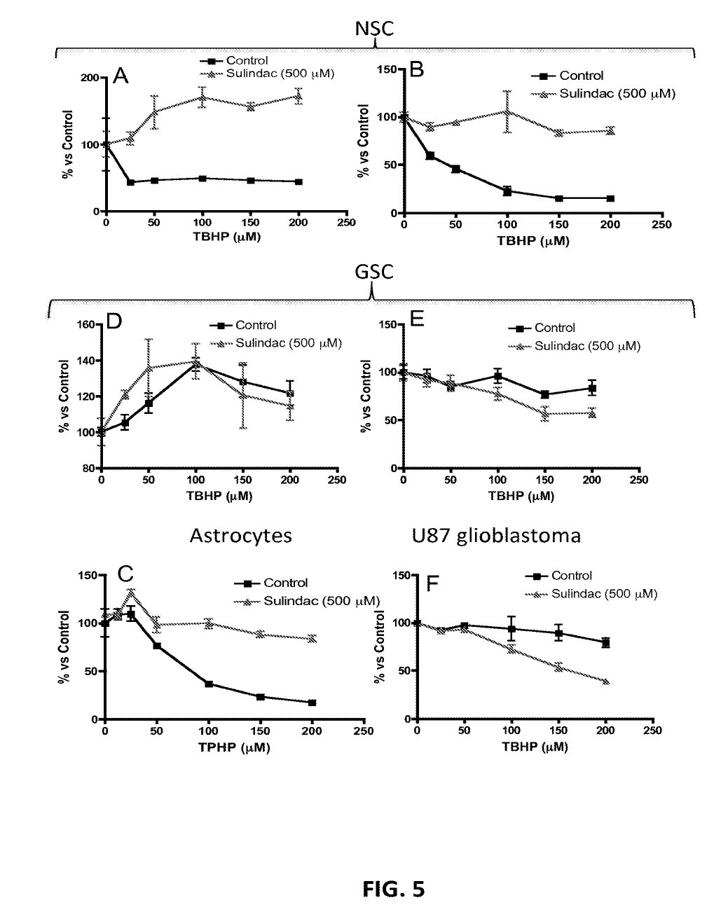

Previous studies have shown a dual effect of sulindac. It can enhance the killing of cancer cells against oxidative stress while protecting normal cells under similar conditions. The sensitivity of normal astrocytes, U87 glioblastoma cells, GSC and NSC to oxidative stress, using TBHP, in the presence or absence of sulindac, was examined. The results are presented in FIG. 5. Using treatment 1 (see methods), the results show that sulindac protected floating NSCs from TBHP-induced death (FIG. 5A). Using treatment 2, sulindac also resulted in complete protection against TBHP-induced cell death on NSCs (FIG. 5B). A very similar protection with sulindac was observed in cultured astrocytes treated for two days with TBHP (FIG. 5C). However, using treatment 1, floating undifferentiated GSCs were very resistant to oxidative stress, and showed no effect of sulindac (FIG. 5D). On the contrary, using treatment 2, where GSC cells should differentiate, the cells showed sensitivity to TBHP and sulindac treatment resulted in increased TBHP-induced cell death as compared with control (FIG. 5E). A similar effect was observed in U87 glioblastoma cells treated with sulindac for 48 hours (FIG. 5F).

These results suggest a protective effect of sulindac against oxidative stress in NSCs and astrocytes with no, or little, effect on floating GSCs. In contrast, sulindac increases sensitivity of U87 cells and differentiated GSCs to oxidative stress, as shown previously with other cancer cell lines. In summary, these results support the hypothesis that GSCs, like other stem cells, are very resistant to oxidative stress, but upon differentiation they behave like U87 cells.

Effect of Sulindac Epimers on GSC Differentiation and Enhanced Killing:

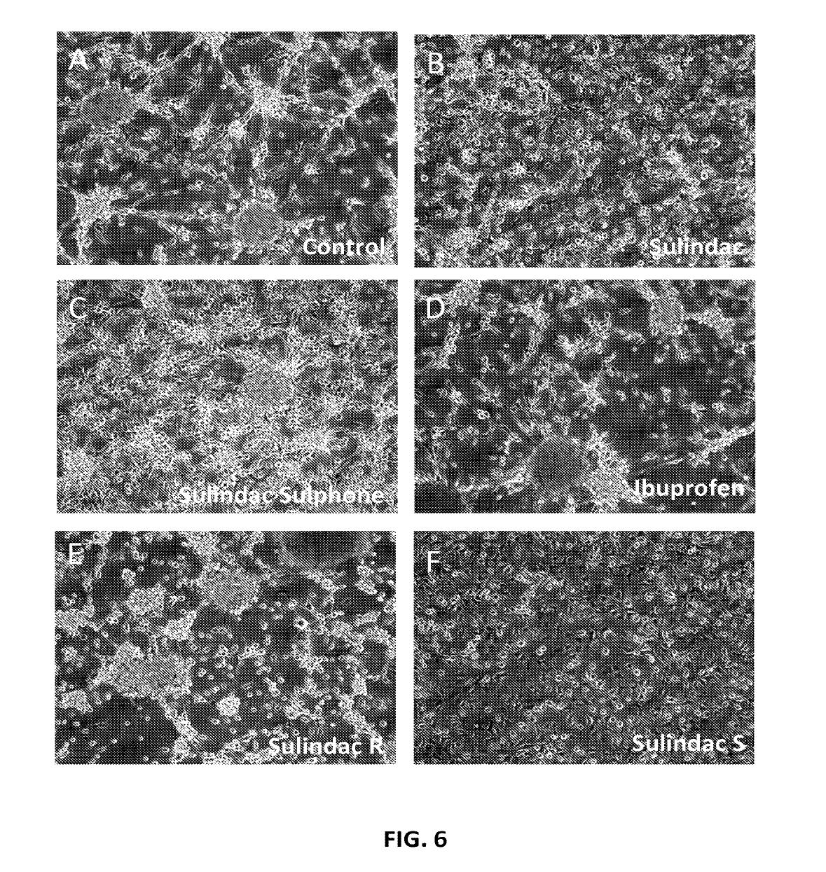

In order to describe the possible mechanisms involved in the sulindac-induced cell differentiation, treatment 2 with sulindac, ibuprofen (another NSAID), sulindac sulphone (a sulindac derivative without NSAID activity) and the R and S epimers of sulindac was used. The two epimers were tested to see whether there was a difference in their ability to differentiate NSCs and GSCs. The results are shown in FIG. 6. The sulindac S-epimer (250 uM) was most potent in inducing cell differentiation, with lower activity observed with sulindac (mixture of R and S epimers) and sulindac sulfone. It should be noted that the sulindac sulfone was used at a much lower concentration because of its toxicity. There was no significant differentiation of GSCs observed with the sulindac R epimer or ibuprofen. These results suggest that the sulindac differentiating effect is not due to its NSAID activity.

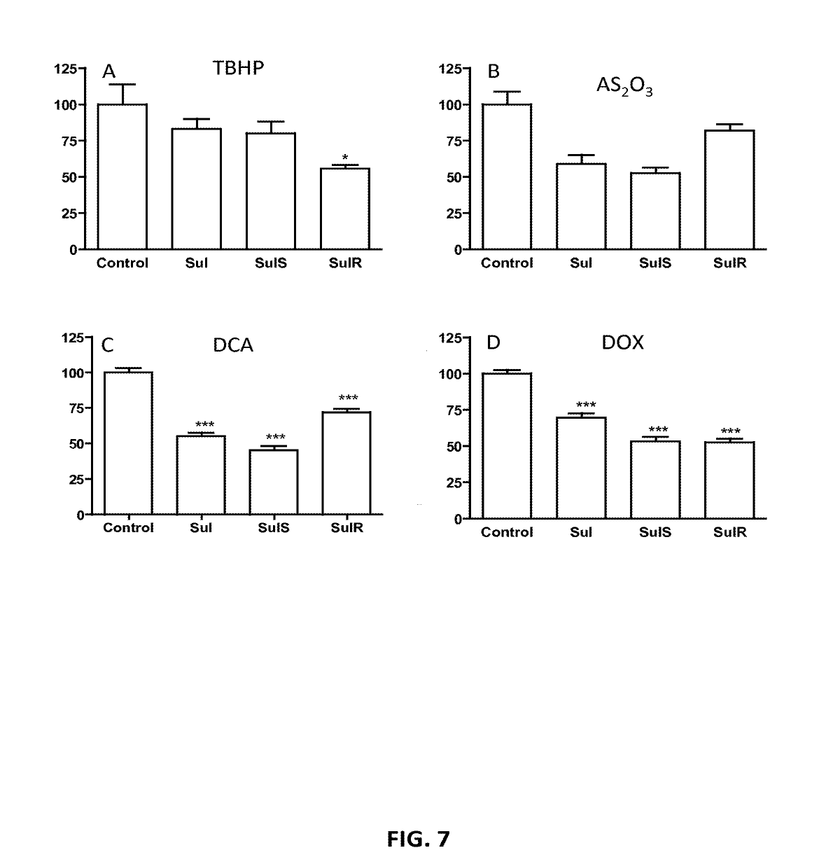

In order to study the effect of the R and S epimers of sulindac in enhancing GSC killing after treatment with different oxidizing agents, treatment 2 with vehicle, sulindac (500 uM) or the S and R sulindac epimers (250 uM) was used. The cells were treated with 200 uM TBHP (FIG. 7A), 3 mM As₂O₃ (FIG. 7B), 30 mM DCA (FIG. 7C) and 400 nM DOX (FIG. 7D) as described in methods. The results show enhanced killing of sulindac with both the S and R epimers in combination with different oxidizing agents. The results with the R epimer were surprising since this epimer did not appear to induce differentiation.

These results confirm previous findings that sulindac can protect normal cells against oxidative stress but enhances oxidative stress-induced killing of cancer cells. In addition, sulindac stimulates cell differentiation on both NSC and GSC and, more importantly, induces higher sensitivity of GSC to oxidative stress.

### Materials and Methods

Cell Cultures:

NSCs were obtained from the hippocampi of PO BL6 mice and cultured in DMEM/F12 medium (Gibco) containing B27 (Gibco), epidermal growth factor (EGF, 20 ng/ml. Invitrogen) and basic fibroblast growth factor (bFGF 10 ng/ml. Peprotech). To induce cell differentiation, NSC where plated on poli-L-lysine (Sigma) were NSC spontaneously differentiate into neurons, astrocytes and oligodendrocytes as previously described (Lopez-Toledano M A & Shelanski M L (2004) J. Neurosci. 24(23):5439-5444).

To obtain a pure culture of astrocytes, NSCs were differentiated in DMEM+10% FBS (both of Gibco) for seven days, trypsinized and re-plated in a new flask with the same medium. Under these circumstances, close to 100% of the cells become astrocytes (GFAP⁺ cells).

GSCs were obtained from the U87 cell line following a protocol previously published. Briefly, the U87 cells were cultured in the same culture medium as NSC. After a few days, floating neurospheres were formed. The floating GSC neurospheres were isolated, mechanically disaggregated and grown in flotation following the same protocol used for NSC. After two passages, a pure culture of floating GSC neurospheres was obtained.

Treatments:

a) Sulindac (Sigma): NSCs were treated as floating neurospheres for 24 hours with vehicle or 500 microMolar sulindac and plated on PLL for another 24 hours before quantification (treatment 1) or plated in PLL for seven days and treated for 48 hours with vehicle or 500 microMolar sulindac (treatment 2). GSCs were treated as floating neurospheres for 24 hours with vehicle or 500 microMolar sulindac and plated on PLL for another 24 hours (treatment 1) or plated in PLL for five days and treated for two days with vehicle or 500 microM sulindac (treatment 2). Astrocytes and U87 cells were plated for seven days and then treated for another 48 hours with vehicle or sulindac. Sulindac was obtained from Sigma. The R and S epimers of sulindac were obtained from Regis Technologies Inc, Morton Grove, Ill. Sulindac R and S were added at a concentration of 250 microM.

b) Oxidizing agents (all of Sigma): TBHP was added for two hours after treatments 1 or 2. Doxorubicin (DOX), dichloroacetate (DCA) and arsenic trioxide (As₂O₃) were added to the cell culture at the same time that sulindac in both treatments 1 and 2. The concentration used were 200 uM TBHP, 3 uM As₂O₃, 30 mM DCA or 400 nM DOX performed in quadruplicate (Error bar: SEM).

Imaging and Immunocytochemistry:
Phase contrast pictures were obtained using an AmScope camera attached to a Nikon TMS microscope. Indirect immunocytochemistry (ICC) was performed as described previously. Briefly, cells were mechanically disaggregated and plated on poly-L-Lysine (PLL). For ICC the following markers were used: Ki67 for proliferative cells; nestin for NSC; beta-tubulin III for neurons; GFAP for astrocytes and 01 for oligodendrocytes. Epifluorescence microscopes (Leica and Nikon) were used for pictures, counting and visualization of the immunocytochemistry. The total number of neurons was quantified by counting a minimum of 15 fields per treatment in triplicates or quadruplicates and normalized vs. non-treated cultures.

Cell Viability Assay:

NSCs, GSCs, astrocytes and U87 cells were plated at 5,000 cells per well in a PLL coated 96-well plate and the cell viability was measured as previously published. Briefly, the cells were grown at 37° C. in a 5% CO₂ incubator for the specified time, the medium discarded under aseptic conditions and replaced with fresh culture medium containing the indicated drug combinations for specified times described in the Results. The culture medium was discarded and the cells were thoroughly rinsed in PBS. Cell viability was determined by using the CellTiter 96 Aqueous One Cell Proliferation Assay (Promega) according to the manufacturer's instructions. Briefly, the assay utilizes a tetrazolium compound that is converted into a water-soluble formazan by the action of cellular dehydrogenases present in the metabolically active cells. The formazan was quantified by measuring the absorbance at 490 nm using a colorimetric microtiter plate reader (SpectraMax Plus; Molecular Devices). Background absorbance was subtracted from each sample. The graphs represent the percentage of cell survival vs. the control without TBHP treatment.

Statistical Analysis:

Analysis of variance (ANOVA) and multicomparison post hoc test (Bonferroni), Student's t test and additional statistics were performed using the Prism4 program from GraphPad Software Inc. The graphs represent the percentage of neurons vs. the control. *p<0.05; **p<0.01; ***p<0.001.

### Example 2

### The S Epimer of Sulindac Induces Cell Differentiation of NSCs and CSCs

Referring to FIG. 3, using the neuronal-specific antibody beta-tubulin III, the percentage of neuronal differentiation vs. total number of cells under two conditions was quantified: 1) Floating NSCs treated for 24 hours with vehicle (A) or 500 microMolar sulindac (C) and plated on PLL for another 24 hours or PLL-plated NSC and treated for 48 hours with vehicle (E) or 2) NSC treated with vehicle (B) or 500 microMolar sulindac at the 7th day post-plating (D, F). Under both conditions, NSC progeny showed higher neuronal differentiation with the sulindac treatment. This experiment demonstrates that sulindac induces neuronal differentiation of NSC.

Referring to FIG. 4, GSCs treated with 500 microMolar sulindac showed a clear morphological differentiation (B, D) vs. their controls (A and C). The Western blot (E) shows a higher neuronal differentiation of GSC after sulindac treatment. This experiment demonstrates that sulindac induces a morphological differentiation on GSC also toward the neuronal phenotype.

Referring to FIG. 5, the effects of the treatment of NSC, astrocytes, U87 cells and GSC with sulindac are shown. Sulindac protected normal non-tumoral cells (NSC and astrocytes) against TBPH-induced death. Sulindac enhanced TBHP-induced cell death in the tumoral line U87 and GSC. This experiment demonstrates that the pretreatment with sulindac enhances GSC sensitivity to oxidative stress.

Referring to FIG. 6, GSCs were treated for two days with A) vehicle, B) 500 uM sulindac, C) 25 uM of sulindac suphone, D) 400 uM of ibuprofen, E) 250 uM of the R epimer of sulindac or F) 250 uM of the S epimer of sulindac after one dp. This experiment demonstrates that only sulindac and its S epimer induces the morphological differentiation of GSC.

Referring to FIG. 7, GSC were treated for 48 hours treatment with vehicle (control), 500 uM sulindac (Sul), 250 uM of the S epimer of sulindac (SulS) or 250 uM of the R epimer of sulindac (SulR). The cells were also treated with A) 200 mM TBHP B), 3 mM As₂O₃, C) 30 mM DCA and D) 400 nM Doxorubicin (DOX). This experiment demonstrates that sulindac and its epimers increase GSC sensitivity to different oxidative agents.

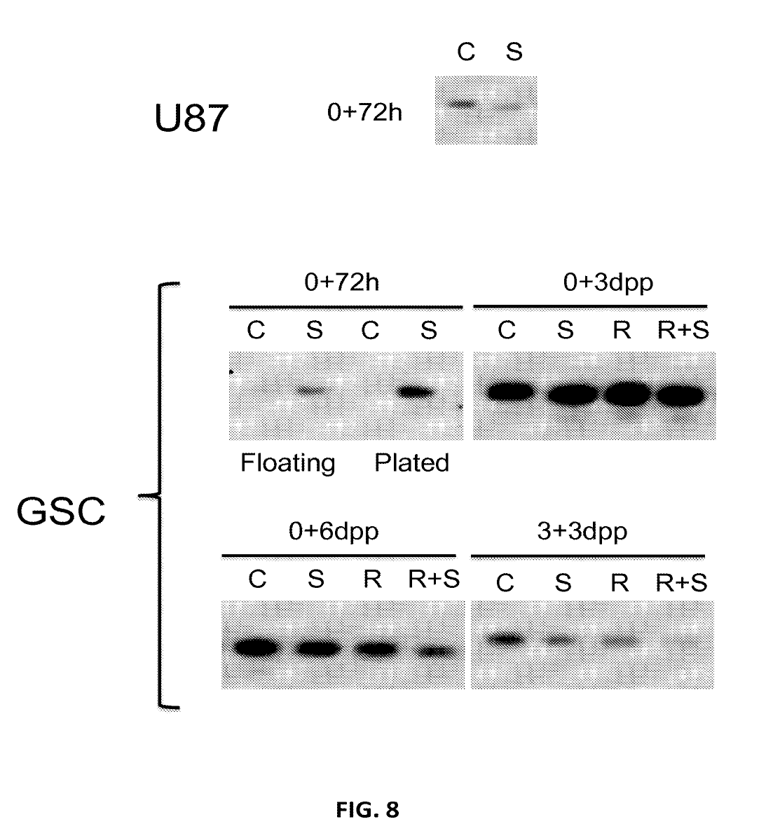

Referring to FIG. 8, changes in the levels of RTP801 in response to sulindac are shown. RRP801, also known as REED1 (Regulated in development and DNA damage response 1), is a hypoxia and stress response gene suppressor of mTOR signaling. In these experiments, whether or not RTP801 could play a role in the sulindac-induced differentiation of GSCs was studied. It was previously shown that RTP801 regulates the timing of cortical neurogenesis both in vivo and in vitro. In the present studies, it was also to be determined if the peroxisome proliferator-activated receptors (PPARs) are involved in the sulindac-mediated differentiation. Rosiglitazone (a PPARγ agonist) induced by itself a partial differentiation. U87, a GBM cell line and GSCs were treated with sulindac, for 72 hours at the same day of plating. GSCs were treated as for 72 hours as floating neurospheres. To test if RTP801 could be involved, GSCs were also treated for 3 and 6 days after plating (0+3d and 0+6d) and for 3 days in the 3rd dpp with sulindac, rosiglitazone and the combination of both. Referring to FIG. 8, sulindac induces a reduction in RTP801 levels in U87 cells. However, it produces an increase in GSCS at 0+72 (both floating and plated), 0+3 and 0+6. When sulindac was added at the 3rd dpp, it induced a decrease in RTP801. These results are consistent with the pattern of differentiation previously shown in NSCS and in vivo (Malagelada et al, 2011, The Journal of Neuroscience, Mar. 2, 2011•31(9):3186-3196) and suggest a possible implication of RTP801 in the differentiating effect of sulindac in GSC.

Because sulindac's differentiating effect was demonstrated in a model of brain cancer, in these experiments, if sulindac can reach the brain and its possible effect in hippocampal neurogenesis was examined 1.67 mg sulindac was injected per mouse during 5 days and the mice were sacrificed 2 hours after the last injection. Six animals were treated with sulindac and six were controls. The levels of sulindac in the brain were measured and immunohistochemistry was performed to see the effect of sulindac in hippocampal neurogenesis. Samples of all other tissues were also saved to test the possible effects of sulindac. Preliminary results show no or very little amount of sulindac in the brain. These experiments are to be repeated and immunocitochemisty is to be performed. While trying to increase the levels of sulindac in the brain, other non-central nervous system cancer stem cells are being studied.

### Example 3

### Future Experiments

Study the effects of sulindac in other CSC types: CSCs can be isolated from lung cancer, breast cancer, skin cancer, pancreatic cancer, prostate cancer and intestine cancer. The effects of sulindac (and the S epimer separately) in cell differentiation in vitro are studied.

Study the effects of sulindac pretreatment in tumor progression in vivo: using nude mice, tumor cells pretreated with sulindac and control are injected into the mice and the tumor size and progression are measured.

Study the effects of sulindac in different cancer stem cells types in vivo: using a well-characterized animal model that express lung cancer, the following is studied:

1) The possible effect of sulindac in preventing tumor formation. The mice are pretreated with sulindac and the time, progression and aggressiveness of the tumor formation as compared with non-treated animals are measured.
2) The effect of sulindac in tumor malignancy: the hypothesis is that sulindac induces cell differentiation and will reduce the ability of the tumor cells to metastasize. Tumors are induced in the animals, the animals are treated with sulindac after the tumor appearance, and the size of the tumor and the possible metastasis after sulindac treated mice as compared with non-treated animals is measured.

### Other Embodiments

Any improvement may be made in part or all of the compositions and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. For example, although the experiments described herein involve GSCs, the compositions and methods described herein can find use in a number of other cancers, including lung cancer, breast cancer, skin cancer, pancreatic cancer, prostate cancer and intestinal cancer. Any statement herein as to the nature or benefits of the invention or of preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method of inducing differentiation of stem cells in vitro comprising culturing stem cells in the presence of sulindac or a sulindac epimer under conditions such that the stem cells differentiate into differentiated cells that can be transplanted into a subject in need thereof.

2. The method of claim 1, wherein the stem cells are cultured in the presence of a sulindac epimer and the sulindac epimer is an S epimer of sulindac.

3. The method of claim 1, wherein the stem cells are neural stem cells (NSCs) and differentiate into neurons.

4. The method of claim 1, wherein the differentiated cells are suitable for use in cell replacement therapy in a subject in need thereof.

5. The method of claim 3, wherein the differentiated cells are neurons, and the subject suffers from a neurodegenerative disease.

6. A method of inducing differentiation of cancer stem cells (CSCs) comprising delivering to a population of cells comprising CSCs a therapeutically effective amount of sulindac or a sulindac epimer for inducing differentiation of the CSCs into cancer cells that are sensitive to oxidative stress.

7. The method of claim 6, wherein the population of cells further comprises NSCs, and the NSCs are protected from oxidative stress.

8. The method of claim 6, wherein the CSCs are glioblastoma stem cells (GSCs).

9. The method of claim 6, wherein the CSCs are lung, skin, breast, liver, intestinal, colorectal, pancreatic or prostate CSCs.

10. A method of increasing sensitivity of CSCs to at least one oxidizing agent or agent that leads to the generation of reactive oxygen intermediates (ROS) comprising delivering to a population of cells comprising CSCs a therapeutically effective amount of sulindac or a sulindac epimer for increasing sensitivity of the CSCs to the at least one oxidizing agent.

11. The method of claim 10, wherein a sulindac epimer is administered, and the sulindac epimer is an R epimer of sulindac.

12. The method of claim 10, wherein the at least one oxidizing agent or agent that leads to the generation of ROS is selected from the group consisting of: As₂O₃, DOX, TBHP, DCA, temozolomide, cisplatin, cyclophosphamide, camptothecin, etoposide, vincristine, methotrexate, gemcitabine, 5-fluorouracil and paclitaxel.

13. The method of claim 10, wherein the population of cells further comprises NSCs, and the NSCs are protected from oxidative stress.

14. The method of claim 10, wherein the CSCs are GSCs.

15. The method of claim 10, wherein the CSCs are lung, skin, breast, liver, intestinal, colorectal, pancreatic or prostate CSCs.

16. A method of increasing killing of CSCs caused by administration of an oxidizing agent or agent that leads to the generation of ROS, comprising administering to a population of cells comprising CSCs a therapeutically effective amount of sulindac or a sulindac epimer for increasing sensitivity of the CSCs to the oxidizing agent or agent that leads to the generation of ROS prior to, concomitant with, or subsequent to administration of the oxidizing agent or agent that leads to the generation of ROS to the CSCs.

17. The method of claim 16, wherein the oxidizing agent or agent that leads to the generation of ROS is selected from the group consisting of: As₂O₃, DOX, TBHP, DCA, temozolomide, cisplatin, cyclophosphamide, camptothecin, etoposide, vincristine, methotrexate, gemcitabine, 5-Fluorouracil and paclitaxel.

18. The method of claim 16, wherein the population of cells further comprises NSCs, and the NSCs are protected from oxidative stress.

19. The method of claim 16, wherein the CSCs are GSCs.

20. The method of claim 16, wherein the CSCs are lung, skin, breast, liver, intestinal, colorectal, pancreatic or prostate CSCs.
